(19) United States
(12) Patent Application Publication
(10) Pub. No.: US 2024/0412860 A1
(43) Pub. Date: Dec. 12, 2024

(54) MEDICAL DEVICE WITH WORKSTATION FUNCTION IN A DATA PROCESSING SYSTEM

(71) Applicant: B. Braun Melsungen AG, Melsungen (DE)

(72) Inventors: Johannes Steube, Knuellwald (DE); Maria Buerger, Malsfeld (DE); Horst Schmoll, Guxhagen (DE); Matthias Paetzold, Melsungen (DE); Klaus Angersbach, Morschen (DE); Stephen Loll, Rotenburg an der Fulda (DE)

(21) Appl. No.: 18/736,160

(22) Filed: Jun. 6, 2024

(30) Foreign Application Priority Data
Jun. 7, 2023 (EP) 23 177 987.7

(51) Int. Cl. G16H 40/63; A61M 5/142

(52) U.S. Cl. CPC G16H 40/63; A61M 5/142; A61M 2205/3561; A61M 2205/3584; A61M 2205/505; A61M 2205/58; A61M 2209/084

(57) ABSTRACT

A medical system includes a medical device, in particular a syringe or infusion pump, with an operating unit/interaction unit, and a data processing unit, in particular a server and/or a digital platform, with an input and/or output unit into which individualized information can be entered. The medical device is actuatable via the individualized information from the data processing unit via a data connection and inputted and transmitted information is displayable at the operating unit. The data processing unit is actuatable via individualized information which can be input into the operating unit and is transmittable to the data processing unit via the data connection. The transmitted individualized information causes an action at the data processing unit.

### CROSS-REFERENCE TO RELATED APPLICATIONS

This application claims priority under 35 U.S.C. § 119 to European Application No. 23 177 987.7, filed on Jun. 7, 2023, the content of which is incorporated by reference herein in its entirety.

### FIELD

The present disclosure relates to a system comprising a medical (treatment) device, in particular a syringe or infusion pump, a data processing unit, and a data connection. In addition, the present disclosure relates to a (medical) designation method, a (medical) notification method, a computer-readable storage medium, and a computer program.

### BACKGROUND

Known medical devices, such as medical pumps, in particular syringe or infusion pumps, can be connected to a (central) data processing unit or a (central) IT platform via a data connection. Several medical devices can be connected to a single (central) data processing unit. The data processing unit may also be a (central) (cloud) server with corresponding hardware for inputting and/or outputting. The data connection can be used, for example, to upload software updates from the data processing unit to the respective medical device or to activate a (software) license on the respective device. On the other hand, the medical devices can transmit therapy data, alarms or warnings to the data processing unit, which are displayed as notifications on an input or output unit of the data processing unit, for example on a screen. In this way, the alarms or warnings of the individual medical devices can be output to a user.

With the known devices, the medical device itself is therefore passive. This means that the medical device can collect data and transmit it to the data processing unit. Furthermore, the medical device can communicate with the data processing unit and provide corresponding outputs to the user. However, it is not possible to actively operate or influence the data processing unit via the medical device (e.g. in the sense of a workstation).

### SUMMARY

It is therefore the object of the present disclosure to overcome or at least reduce the disadvantages of the prior art and in particular to provide a medical system, a (medical) designation method, a (medical) notification method, a computer-readable storage medium, and a computer program which enables active control of a (central) data processing unit in particular on a medical device of the above-mentioned type (as a workstation).

These objects of the present disclosure are solved with respect to a medical system according to the disclosure, with respect to a (medical) designation method according to the disclosure, with respect to a (medical) notification method according to the disclosure, with respect to a computer-readable storage medium according to the disclosure, and with respect to a computer program according to the disclosure.

The present disclosure relates to a medical system comprising at least a medical device, in particular a syringe or infusion pump, with an operating unit/interaction unit, a (central) data processing unit, in particular a (cloud) server or a digital platform, and a data connection which connects the medical device and the data processing unit. The data processing unit has an input and, if applicable, output unit into which (device-) individualized information can at least be entered/input. The (device-) individualized information is transmittable via the data connection from the data processing unit to the at least one (selected) medical device, and the medical device is (individually) actuatable by the data processing unit on the basis of the entered and transmitted information. The operating unit of the medical device is furthermore prepared to display the information entered and transmitted to the data processing unit. Furthermore, the medical system is prepared to transmit individualized (control) information that can be entered or is entered into the operating unit (straight/directly or via a transportable, possibly wirelessly connected input device such as a tablet, laptop, etc.) to the data processing unit via the data connection, wherein the input of the individualized information into the operating unit causes an (influencing) action at the data processing unit (i.e. the data processing unit is reprogrammed, for example, in such a way that it performs a modified control of at least this one individual device from then on).

Accordingly, device-individualized information means, for example, a device recognition, a control command or a control parameter or the like, which relates exclusively to this one medical device (possibly among several medical devices) and which has to be stored accordingly in the data processing unit. An effected action is to be understood as any process on the part of the data processing unit that is triggered by the input on the at least one medical device, for example the saving of a parameter or device state, the changing of a control parameter or of a control step and the like.

In other words, the medical (treatment) device preferably has an input and, if applicable, output unit or an (individual) interface to a device-related (mobile) input and, if applicable, output device (tablet, laptop, cell phone, etc.) for (manual) inputting and, if applicable, outputting of information or data into the medical (treatment) device. The data processing unit, or central server, or central computer has hardware with the input and/or output unit for inputting and/or outputting information into the data processing unit itself. Information that can be entered into the input and/or output unit of the data processing unit can be transmitted to the medical device via the (systems own) data connection (different from the individual interface for a mobile input device that may be present on the treatment device side), which in particular may be a wireless data connection such as WIFI, Ethernet and/or Bluetooth and/or a cable-based bus system such as USB. The medical device can therefore be actuated/controlled by the inputs on the data processing unit itself. For example, (software) updates can be sent to the medical (treatment) device via the data processing unit, and the (treatment) device, e.g. pump, can thus be updated (wirelessly or remotely) via the data processing unit. Information that is entered into the input and/or output unit of the respective medical device can be transmitted (in the opposite direction) from the medical device to the data processing unit via the data connection. This means that the data processing unit can also be actuated via these inputs on the medical (treatment) device. The inputs on the medical device or on the operating unit (input and, if applicable, output device) of the medical device are processed as a command by the data processing unit. In particular, a defect in the medical device, in particular the medical pump, can be reported by inputting information to the medical device. The defect notification/defect information may trigger/initiate an (influencing) action at the data processing unit, which, for example, initiates corrective maintenance. The (central) data processing unit can therefore be controlled by the information input on the medical device.

In yet other words, the respective input and/or output devices/operating units (respectively) on the at least one (decentralized) medical (treatment) device and also on the (central) data processing unit as well as the data connection between the medical device and the data processing unit, thus enable active bidirectional (i.e. acting in both directions) communication between the medical device and the (central) data processing unit as a central (digital) platform. The data processing unit is therefore actuatable via the (integral or separate) operating unit of the respective medical (treatment) device in a similar way to a remote control and/or with an active messaging and communication service.

A core of the disclosure thus lies in the fact that the data processing unit as the central platform can be controlled via the respective medical (treatment) device, such as a pump in the function of a (satellite) workstation, in that inputs which are made on the respective medical (treatment) device can be transmitted to the central data processing unit as the platform and the data processing unit is actuated via these inputs.

The medical device may in particular be a medical pump, preferably a syringe or infusion pump. However, the medical device may also be another medical instrument or device such as a medical robot, an endoscope, a monitoring device such as a blood pressure monitor, a heart rate monitor, a medical ventilator or an extracorporeal blood treatment machine. The medical device may in turn be a device that is built into another/superordinate medical device. For example, the medical device may be a pump, possibly an infusion pump, syringe pump, delivery pump, etc., whereas the other, superordinate medical device may be, for example, an extracorporeal blood treatment machine, heart-lung machine or similar devices in which the medical device is installed/connected. It is only relevant that the medical device can have an (integral or mobile, satellite-like) operating unit as input and, if applicable, output device and a (data) interface with the data processing unit or can be coupled/is coupled with such a decentralized operating unit, wherein the medical device or its (data) interface is provided to transmit (control) information entered at the operating unit also in the direction of the central data processing unit.

This has the advantage over the prior art that the operating unit of the, preferably each or selected, (decentralized) medical device is configured to be used/usable for at least limited or full active influencing (programming) of the (central) data processing unit, preferably in addition to or even as an alternative to its own input and, if applicable, output unit.

In particular, the operating unit may be a (touch) screen on the medical device. Furthermore, the medical device may also have input buttons or knobs via which the user can make inputs to the medical device. If the medical device is a medical pump, then the (touch) screen can be arranged in particular in a front flap of the pump. The input buttons may be arranged next to the screen. The input buttons may also be one or more operating elements. Furthermore, the operating unit may also have lamps via which signals can be output or displayed in particular.

The data processing unit may in particular be a server or a computer which is connected (in a data transferable manner) to the number, preferably plurality, of medical devices such as pumps. In particular, the data processing unit may have a data processing unit's own (central) input and/or output unit to provide inputs and outputs (directly/straight) to the server or computer. The input and/or output unit may, for example, be a combination of mouse, keyboard and a screen and/or a touchscreen. Voice-controlled input and output is also conceivable.

The data connection (of the system) may be a WIFI and/or an Ethernet connection in particular. Of course, a (wireless) data connection via Bluetooth, NFC, and/or a wired data connection via USB or another bus system is also possible.

The individualized information may be, for example, identification information of the medical device, in particular of the medical pump, such as a serial number and/or a model number of the medical device, in particular of the pump. The identification information enables the data processing unit to identify a wanted medical device connected to the data processing unit. The individualized information may further be defect information of the medical device. The defect information may be a type of defect, the serial number of the defective medical device, a repair instruction and/or a request to alert a user or to lock the device.

Furthermore, the individualized information may also be detectable by scanning a computer-readable code, for example a QR code or a barcode. By scanning a medical product or medical device, for example, the correct product or device can be identified. The identified product or device can then be actuated (by the data processing unit).

Bidirectional communication allows the data processing unit to be controlled by the medical device. For example, the defect on the medical device can be reported. The defect message can trigger an action at the data processing unit to initiate a repair or corrective maintenance of the medical device. Bidirectional communication can also be used to send information from the data processing unit to the medical device. For example, a specific device can be selected from a plurality of devices and can be made recognizable.

The object of the present disclosure is further solved by a (medical) designation method (in the sense of labeling, marking) of a defined/predetermined/to be selected/wanted medical (treatment) device from a plurality of medical (treatment) devices, wherein the medical devices are each connected to the data processing unit via the (system's own) data connection. The designation method according to the disclosure comprises the following steps:

Device-specific information, in particular a device ID and/or a model number and/or serial number of a medical device and/or other device information, is entered into the input and/or output unit of the data processing unit itself. The wanted medical (treatment) device is selected or identified by the data processing unit based on the entered (device-specific) information. In order to identify the wanted medical device, the input (treatment) device-specific information can be linked in particular in a database with information about the respective medical devices. The (central) data processing unit controls the wanted medical device (via the systems own data connection). Due to the actuating of the wanted medical device by the data processing unit, a designation or marking perceptible by the user is output via an operating unit, in particular via the (touch) screen of the wanted medical device, whereby the wanted medical device is recognizable/identifiable (by the user) in the plurality of medical devices. The wanted medical device can thus be designated and/or marked and made identifiable by the perceptible designation or marking in differentiation from the other medical devices.

The number or plurality of medical devices is therefore connected to the (central) data processing unit via the data connection. The medical devices in particular can be registered with the data processing unit in such a way that the individual medical devices are identifiable and actuatable by the data processing unit. The identification of the individual medical device in particular can take place via an assignment of cables that connect the medical devices to the data processing unit or via an electronic identification or assignment procedure. The data processing unit may thus be suitable for controlling the individual connected medical devices individually.

Specifically, a user can select a desired medical device on the data processing unit. To do this, the user can, for example, press a button on the input and/or output unit of the data processing unit or can enter a device ID that is assigned to a specific medical device. The data processing unit can control the desired medical device based on the user's input.

The controlled medical device can then output the marking or designation, which can be perceived by the user visually, acoustically and/or haptically. Preferably, the operating unit of the medical device can emit an optical signal such as a flashing screen and/or an acoustic signal such as a beep or whistle. It may also be possible for the medical device and/or the operating unit to vibrate. In principle, any signal that can be perceived by the user and that sets the wanted device apart or distinguishes it from other medical devices is conceivable.

In particular, the method allows the wanted medical device to be found and identified quickly and easily by the user. Thus, for example, a defective device or a device that requires maintenance can be recognized quickly.

Furthermore, the present disclosure relates to a (medical) (defect) notification method for notifying/indicating a defect in a medical device. Defect information about a defect in the medical device is entered into the operating unit of the medical device in which the defect has occurred. The entered defect information is transmitted to the data processing unit via a data connection, which may be part of the medical system. The transmitted defect information triggers an action at the data processing unit. The transmitted defect information can also be output (to the user) via an input and/or output unit of the data processing unit in order to indicate a defect in the medical device.

Preferably, the action of the data processing unit may consist of initiating a repair or corrective maintenance of the medical device on the basis of the transmitted defect information. For example, a technician can be notified or informed (automatically or initiated by the data processing unit) to repair the medical device.

Furthermore, the action at the data processing unit may also consist of entering the reported defect in a database of the data processing unit. As a result, the data processing unit can store or record that the medical device is defective. The data processing unit can thus indicate the defect to the user if the medical device in question is selected or actuated at a later time. Furthermore, the defective medical device can be blocked by the data processing unit in such a way that it cannot be used until it has been repaired. At the very least, when using the defective device it can warn that it is defective.

With the (medical) notification method, the user can, for example, enter the defect directly on the medical device and can instruct the repair. This can make input of the defect at the data processing unit superfluous. Manual instruction of the repair or corrective maintenance by the user can therefore also be omitted.

According to an optional aspect of the present disclosure, the individualized (or also standardized) information that can be entered into the input and/or output unit of the data processing unit may be recognition information or device-specific information via which the wanted medical device is identifiable or actuatable by the data processing unit. The data processing unit can therefore use the information entered to identify and actuate the wanted medical device and to make it recognizable in the plurality of medical devices.

In other words, the device-specific information by which the medical device is identifiable can be input (by the user) to the input and/or output unit of the data processing unit. The entered information can be processed by the data processing unit. Based on the device-specific information, the data processing unit can identify the medical device wanted by the user and can actuate the wanted identified medical device.

Preferably, the data processing unit has a (stored) databank in which the device-specific information, such as a device ID, is assigned to a specific medical device. The data processing unit can be adapted to detect and control the specific/wanted medical device based on the entered device ID. Optionally, the entered recognition information can be transmitted to the detected medical device via the data connection. Thus, the data processing unit can determine and actuate the wanted medical device based on the device ID in order to mark it in the plurality of other medical devices.

According to a further optional aspect of the present disclosure, the medical device may be actuated by the data processing unit in such a way that a designation of the wanted medical device is output via the operating unit (of the detected/wanted medical device). Preferably, the designation is output via the operating unit, for example by flashing of the operating unit or displaying graphical information on the operating unit. In particular, a (colored) margin around the operating unit can light up or flash. This allows a user to identify the wanted medical device quickly and easily. This can be particularly helpful if a single medical device is to be identified in a set/plurality of medical devices. Optionally, the transmitted recognition information about the operating unit (of the detected/wanted medical device) may be displayable. Furthermore, the wanted device may emit other optical, acoustic or haptic signals.

Preferably, the individualized information that can be entered into the operating unit may be device-state information, in particular defect information, which indicates a (current) state of the medical device. In particular, the device-state information can indicate that the medical device has a defect and/or requires maintenance. The device-state information may be transmittable to the data processing unit via the data connection and may cause an action at the data processing unit. For example, the user may set or enter directly on the medical device that there is a defect. The data processing unit processes this information entered by the user independently. The action at the data processing unit may be, for example, the automated instruction to repair the device and/or block the device.

However, the device-state information may also be an indication of upcoming (scheduled or regular) maintenance or cleaning of the medical device. In this case, the action of the data processing unit may consist of instructing the maintenance or cleaning or entering it in a calendar.

Preferably, the device-state information transmitted to the data processing unit may be displayable at the input and/or output unit of the data processing unit. The device-state information may be in particular the defect information about the required maintenance or repair of the medical device.

According to a further optional aspect of the present disclosure, the data processing unit may be adapted to switch off, lock and/or initiate repair/corrective maintenance of the medical device upon receipt of the defect information. The data processing unit can thus ensure that the medical device is no longer usable in a detected defective state and automatically or automated causes the defective device to be repaired.

Preferably, the operating unit of the medical device may be a touchscreen. The user's inputs on the touchscreen may display the defect information. The inputs, in particular the defect information, may cause the action at the data processing unit. If the inputs are the defect information, the action in particular may be to initiate the repair of the medical device. This means that the user no longer has to manually initiate the repair of the medical device.

Preferably, the system may have a carrier system into which a plurality of the medical devices can be inserted. The data processing unit may be arranged in the carrier system. This means that the data processing unit may be arranged in an existing component. The system may make use of an existing connection between the individual devices and the carrier system, for example for the power supply or for the transmission of sensor data.

The objects of the present disclosure are solved with respect to the computer-readable storage medium and with respect to the computer program in that they comprise instructions which, when executed by a computer, cause the computer to perform the medical procedures according to the present disclosure.

Any disclosure related to the medical system according to the present disclosure applies equally to the medical methods according to the present disclosure, in particular by analogy, as well as vice versa.

### BRIEF DESCRIPTION OF THE DRAWINGS

The disclosure is explained in more detail below based on a preferred configuration example with reference to the accompanying Figures.

FIG. 1 shows a schematic representation of a medical system according to a preferred embodiment of the present disclosure;

FIG. 2 shows a perspective view of a medical pump as a medical device according to the present disclosure;

FIG. 3 shows an illustration of an operating unit of the medical device according to the present disclosure;

FIG. 4 shows a further illustration of the operating unit of the medical device according to the present disclosure;

FIG. 5 shows a perspective view of a medical system according to an embodiment of the present disclosure;

FIG. 6 shows a flowchart for a medical designation method according to a preferred embodiment of the present disclosure; and

FIG. 7 shows a flowchart for a medical notification method according to a preferred embodiment of the present disclosure.

### DETAILED DESCRIPTION

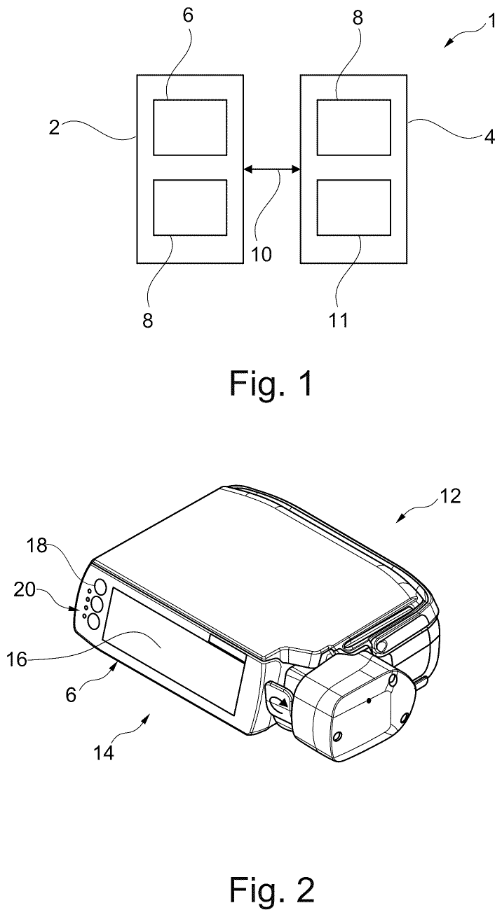

FIG. 1 shows a medical (control/monitoring/checking) system 1 with at least one (decentralized, possibly mobile) medical (treatment) device 2 and a (central, possibly stationary) data processing unit 4. The medical device 2 is preferably a medical syringe or infusion pump. The medical device 2 has its own operating unit 6 for input and, if applicable, output of information or is coupled with such a device-own (mobile) operating unit 6 via a device-internal data connection (cable, Bluetooth, etc.). The data processing unit 4 also has an input and, if applicable, output unit 8. Inputs and, if applicable, outputs to the data processing unit 4 can be made via the input and, if applicable, output unit 8. The data processing unit 4 and the medical device 2 are connected to each other via a data connection 10, preferably as a further component of the medical system 1, in such a way that data and/or information can be transmitted from the at least one medical device 2 to the data processing unit 4 and vice versa and preferably from one medical device 2 to another medical device.

The data processing unit 4 may preferably be a (central) server, a computer, a digital platform or a central control unit. The data processing unit 4 has or is coupled to a storage unit in which a database is stored or saved.

Instead of the aforementioned pumps, the medical device 2 may also be a surgical robot, a robot-assisted visualization unit and/or another medical robot with an operating unit 6.

In particular, the data connection 10 may be a wired or wireless data connection via WIFI, Bluetooth and/or NFC and/or a wired data connection via USB, Ethernet or another bus system.

FIG. 2 shows the medical device 2 in an exemplary embodiment. This embodiment is a medical syringe pump 12. In a generally known manner, the syringe pump 12 has a front flap 14 through which a syringe (not shown) can be fixed in the syringe pump. The front flap 14 also has the operating unit 6, or the operating unit 6 may be integrated into the front flap 14. The operating unit 6 has a screen 16, (input) buttons 18 and further lamps 20. The screen 16 may be configured as a touch screen so that inputs and outputs may be made via the screen 16. Inputs for the syringe pump 12 can be made via the buttons 18.

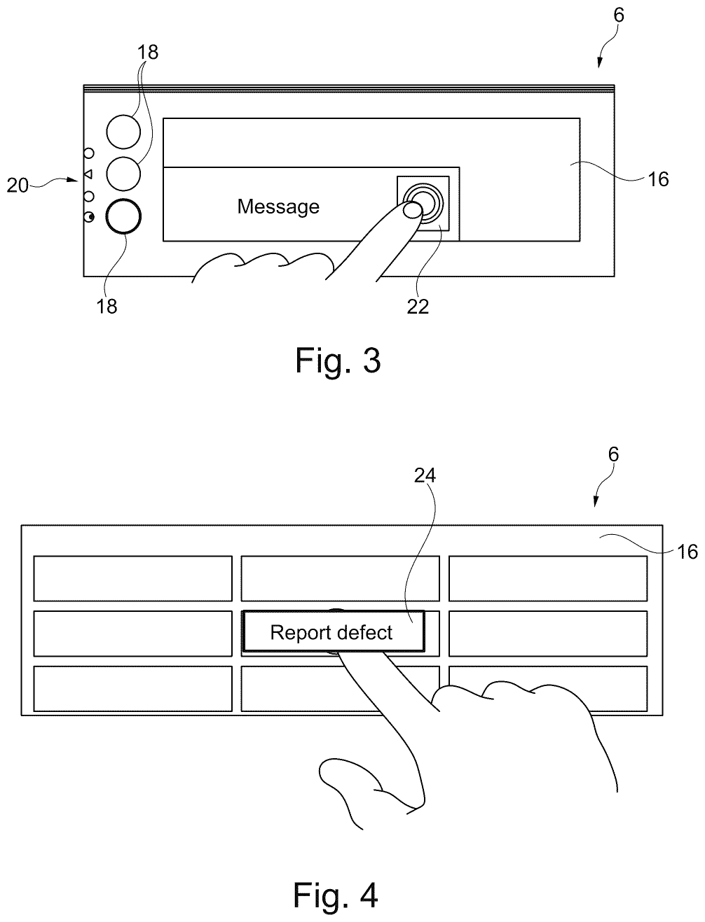

FIG. 3 shows the operating unit 6 in detail. The operating unit 6 has the screen 16, the input buttons 18 and the lamps 20. A dialog window 22 can light up on the screen 16 to indicate to a user that a notification has been transmitted by the data processing unit 4. The notification from the data processing unit 4 may be, for example, that the medical device 2, which is already registered in the data processing unit 4 as a peripheral device, has been selected (by the user) in the data processing unit 4. If the medical device 2 has been selected in the data processing unit 4, the notification is displayed, for example, on the screen 16 of the medical device 2, or another signal is triggered (lamp, signal tone, etc.), which marks this one medical device 2 so that the user can identify the selected medical device 2 or can verify the selection of the correct device on the selected device itself. In this case, the operator therefore does not have to rely solely on the output on the central data processing unit.

FIG. 4 also shows the screen 16 of the operating unit 6. Another dialog window 24 is displayed on the screen 16. The dialog window 24 has the text “Report defect”, for example. If the dialog window 24 on the touch screen 16 is activated by the user, this defect information is transmitted to the data processing unit 4. The transmitted defect information then triggers the action at the data processing unit 4. The action may be, for example, that the data processing unit 4 instructs or orders the repair of the medical device 2.

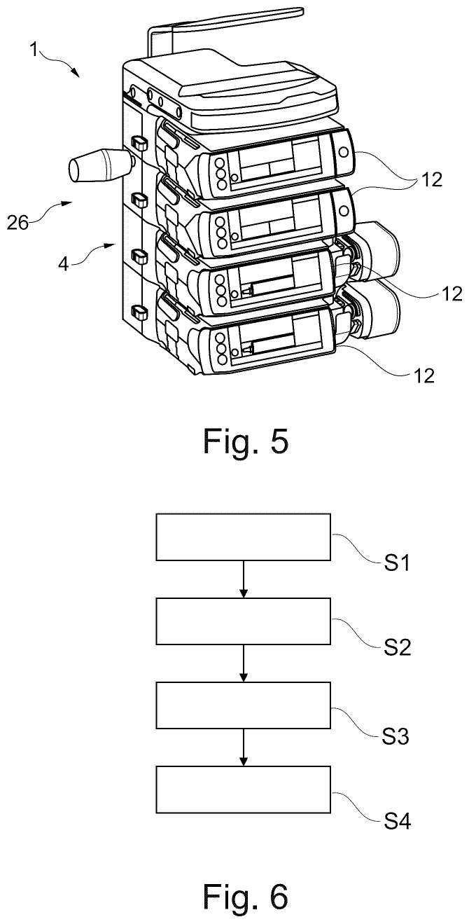

FIG. 5 shows an arrangement of several medical devices in the form of pumps 12 in a common carrier system (device rack) 26. The carrier system 26 may have the data processing unit 4. This has the advantage that the carrier system 26 is already present and comprises an electrical and data-transmitting connection with a number of medical pumps 12. In this case, the central data processing unit 4 and the data connection 10 are therefore installed/integrated in the carrier system 26 or mounted on it and thus, together with the carrier system 26, may be mobile, whereas the medical devices 12 are basically mobile with respect to the carrier system 26 and can be inserted into the carrier system 26 as required and coupled to the data connection.

FIG. 6 shows a flow chart for a (medical) designation method (in the sense of the above definition) of a defined/predetermined/to be selected/wanted medical device 2 from a plurality of medical devices 2. The individual medical devices 2 are each connected to the data processing unit 4 via the data connection 10 (and already registered), so that data or information can be transmitted from the data processing unit to the individual medical devices 2.

In step S1, a user enters device-specific information directly into the input and, if applicable, output unit 8 of the data processing unit 4, for example. The device-specific information can be used to identify the specific wanted medical device 2 from the plurality of medical devices 2. The device-specific information may be, for example, the serial number of the wanted medical device 2.

In step S2, the data processing unit 4 selects the wanted medical device 2 from the plurality of medical devices 2 on the basis of the device-specific information entered. This means that the wanted medical device 2 is identified by the data processing unit 4 on the basis of the transmitted device-specific information, in particular the serial number.

In step S3, the data processing unit 4 controls the wanted and now identified/detected medical device 2 via the data connection 10.

In step S4, a designation or marking is output via the operating unit 6 of the wanted/detected medical device 2, i.e. the wanted/detected medical device 2 can be designated/marked, for example by the selected/wanted medical device identifying itself via a signal. In particular, the designation may be a graphic marking such as flashing on the screen 16 of the operating unit 6 of the medical device 2. For example, the screen 16 may be outlined with a red frame that flashes when one of the medical devices 2 has been identified as the wanted medical device 2. Furthermore, the lamps 20 of the medical device 2 may light up or flash to designate the medical device 2.

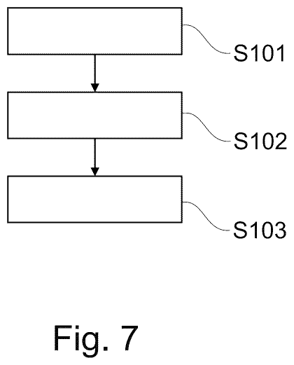

FIG. 7 shows a (medical) (defect) notification method in the event of a defect in the medical device 2. The notification method has the following steps. In step S100, a user (manually) enters defect information into the operating unit 6 of the medical device 2 on which a defect has occurred. The defect of the medical device 2 may be recognized by the user and/or the device 2 itself may indicate or report that there is a defect. In step S101, the entered defect information is transmitted from the medical device 2 to the data processing unit 4 via the data connection 10. In step S102, the transmitted defect information causes an action on the data processing unit 4. The transmitted defect information may also be output by the input and/or output unit 8 of the data processing unit 4 (to the user) in order to indicate the defect on the medical device 2. In particular, the defect information may be displayed by a dialog window on the input and/or output unit 8 of the data processing unit 4. The defect information may be used, for example, to output the serial number of the defective device 2, information about the defect and/or whether corrective maintenance is necessary. Furthermore, the defect information may be used to independently instruct technical personnel to repair or perform corrective maintenance on the medical device 2 or to block the defective device 2 for use. The data processing unit 4 may store or save the information about the defect of the medical device 2 in the databank.

### LIST OF REFERENCE SIGNS

1 system
2 medical device
4 data processing unit
6 operating unit
8 input and/or output unit of the data processing unit
10 data connection
11 storage unit
12 syringe pump
14 front flap
16 screen
18 input buttons
20 lamps
22 first dialog window
24 further dialog window
26 carrier system
S1 Step 1
S2 Step 2
S3 Step 3
S4 Step 4
S101 Step 101
S102 Step 102
S103 Step 103

1.-15. (canceled)

16. A medical system comprising:
a medical device; and
a data processing unit,
the medical device and the data processing unit being connected by a data connection,
the medical device comprising an operating unit,
the data processing unit comprising an input unit, wherein device-individualized information is enterable into the input unit,
the medical device being actuatable by the data processing unit via the data connection based on the device-individualized information,
the data processing unit being actuatable via the device-individualized information, which is entered into the operating unit of the medical device and is transmitted to the data processing unit via the data connection, and
wherein the device-individualized information causes an action at the data processing unit.

17. The medical system according to claim 16, wherein information, which is entered into the data processing unit and is transmitted to the medical device, is displayed on the operating unit.

18. The medical system according to claim 16, wherein the device-individualized information, which can be entered into the input unit of the data processing unit, is device-specific information, via which a wanted medical device of a plurality of the medical devices is identifiable via the data processing unit.

19. The medical system according to claim 18, wherein the device-specific information is a device ID, a model number and/or a serial number.

20. The medical system according to claim 18, wherein the data processing unit comprises a databank,
wherein the device-specific information is assigned to a specific medical device in the databank, and
wherein the data processing unit is adapted to detect the wanted medical device and to transmit recognition information to the detected medical device based on the entered device-specific information.

21. The medical system according to claim 20, wherein the detected medical device is actuatable by the data processing unit in such a way that the medical device outputs a designation via the operating unit.

22. The medical system according to claim 21, wherein the designation is a flashing of the operating unit and/or graphic information being displayed on the operating unit and/or a colored margin around a pop-up field displayed on the operating unit.

23. The medical system according to claim 16, wherein the device-individualized information which can be entered into the operating unit is device-state information, which depicts a current state of the medical device, and
wherein the device-state information is transmittable to the data processing unit via the data connection and the device-state information, once transmitted, causes the action at the data processing unit.

24. The medical system according to claim 23, wherein the device-state information is defect information.

25. The medical system according to claim 23, wherein the current state of the medical device comprises a defect of the medical device and/or a need of the medical device to be serviced.

26. The medical system according to claim 23, wherein the device-state information transmitted to the data processing unit is displayable at the input unit of the data processing unit.

27. The medical system according to claim 23, wherein the data processing unit is adapted to switch off, to lock and/or to initiate repair and/or maintenance of the medical device upon receipt of the device-state information.

28. The medical system according to claim 16, wherein the operating unit of the medical device is a touch screen and inputs of a user on the touch screen cause the action at the data processing unit.

29. The medical system according to claim 16, wherein the medical system comprises a carrier system into which a plurality of medical devices is insertable and wherein the carrier system comprises the data processing unit.

30. The medical system according to claim 16, wherein the medical device is a syringe or infusion pump.

31. The medical system according to claim 16, wherein the data processing unit is a cloud server.

32. A medical designation method of a selected medical device in a plurality of medical devices, each of which is connected to a data processing unit via a data connection, the medical designation method comprising the steps of:
inputting device-specific information into an input and/or output unit of the data processing unit;
selecting a wanted medical device by the data processing unit based on the device-specific information, in which the device-specific information is linked to device information;
driving of the selected medical device by the data processing unit via the data connection; and
outputting a user-perceivable designation by an operating unit of the selected medical device, whereby the selected medical device is recognizable in the plurality of medical devices.

33. The medical designation method according to claim 32, wherein the user-perceivable designation is an optical, acoustic and/or haptic signal and/or a flashing of a screen of the operating unit, beeping of the wanted medical device, and/or vibration of the wanted medical device.

34. A computer-readable storage medium comprising instructions which, when executed by a computer, cause the computer to perform the steps of the medical designation method according to claim 32.

35. A computer program comprising instructions which, when executed by a computer, cause the computer to perform the steps of the medical designation method according to claim 32.

36. A medical notification method for a medical device comprising the steps of:
inputting defect information into an operating unit of a medical device on which a defect has occurred;
transmitting the defect information from the medical device to a data processing unit via a data connection; and
triggering an action at the data processing unit based on the defect information.

37. The medical notification method according to claim 36, wherein the action at the data processing unit initiates a repair of the medical device when a defect in the medical device has been detected, and/or locks the medical device.

38. A computer-readable storage medium comprising instructions which, when executed by a computer, cause the computer to perform the steps of the medical notification method according to claim 36.

39. A computer program comprising instructions which, when executed by a computer, cause the computer to perform the steps of the medical notification method according to claim 36.
